(19) United States
(12) Patent Application Publication
(10) Pub. No.: US 2026/0151619 A1
(43) Pub. Date: Jun. 4, 2026

(54) MICROCURRENT APPLICATION APPARATUS FOR WOUND HEALING

(71) Applicant: BIOLAMP Co., Ltd., Jeonju-si (KR)

(72) Inventors: MinSik SEO, Gwangju (KR); Soyang KIM, Jeonju-si (KR); HyukJin SEO, Gwangju (KR); Youngsoon LEE, Jeonju-si (KR)

(21) Appl. No.: 19/310,830

(22) Filed: Aug. 26, 2025

(30) Foreign Application Priority Data
Dec. 4, 2024 (KR) 10-2024-0178401

(51) Int. Cl. A61N 1/04; A61F 13/00; A61F 13/0203; A61F 13/0246; A61N 1/36

(52) U.S. Cl. CPC A61N 1/0468; A61F 13/0226; A61F 13/0246; A61N 1/0476; A61N 1/0492; A61N 1/36031; A61F 2013/00948

(57) ABSTRACT

Proposed is a microcurrent application apparatus for wound healing. The microcurrent application apparatus includes a healing pad part attached to a wound site, a pH sensor attached to the healing pad part and configured to measure a pH value of the wound site, at least one electrode attached to the healing pad part and configured to apply a microcurrent to the wound site, and a current controller configured to control at least one of polarity and intensity of the microcurrent applied through the electrode on the basis of the pH value measured by the pH sensor. Through this configuration, the severity or current condition of a wound is objectively determined by detecting the pH value, and accordingly an appropriate microcurrent is applied, thereby maximizing the therapeutic effect.

### CROSS REFERENCE TO RELATED APPLICATION

The present application claims priority to Korean Patent Application No. 10-2024-0178401, filed Dec. 4, 2024, the entire contents of which are incorporated herein for all purposes by this reference.

### BACKGROUND OF THE INVENTION

### Field of the Invention

The present disclosure relates generally to a microcurrent application apparatus for wound healing. More particularly, the present disclosure relates to a microcurrent application apparatus for wound healing, which is attached to a wound site and treats the wound by applying microcurrents.

### Description of the Related Art

A wound healing process is largely divided into the following four phases: a coagulation phase (phase 1), an inflammatory phase (phase 2), a proliferative phase (phase 3), and a remodeling phase (phase 4).

Among these healing stages, the proliferative phase is the period during which cells constituting the skin proliferate to restore original appearance thereof. During this phase, regeneration and covering of the lost epidermis (reepithelialization), regeneration of blood vessels (angiogenesis), and repair of damaged tissue (fibroplasia) take place.

Specifically, reepithelialization involves a series of processes, including migration of epidermal cells, proliferation of epidermal cells, contact inhibition, and restoration of skin thickness. The migration of epidermal cells begins approximately 24 hours after a wound occurs.

Under normal conditions, epidermal cells are tightly attached to one another. However, when a wound occurs, various signals are exchanged, and protein-decomposing enzymes are secreted to break intercellular bonds, and the epidermal cells migrate from the edge of the wound to the wound site. These migrating cells have suppressed proliferation ability, which means that the cells rapidly migrate to the wound site, cover the damaged area, and then increase in number. Epidermal cell proliferation is the period when cells behind the migrating epidermal cells around the wound undergo mitosis to replace the basal cells, and these basal cells migrate toward the area of the skin defect.

Contact inhibition occurs when the most advanced epidermal cells that were migrating stop moving after becoming completely surrounded by epidermal cells migrating from the opposite edge of the wound. At this point, the surrounding epidermal cells begin to differentiate again, forming new skin.

Finally, restoration of skin thickness is the process in which epidermal cells continue to divide until normal skin thickness is restored after connection between the epidermal cells is made.

Recently, a technology that assists in wound treatment by applying microcurrents to a wound site has been disclosed. For example, in Korean Patent No. 10-2525070 titled “OPERATING METHOD USING MICROCURRENT STIMULATION APPARATUS FOR WOUND TREATMENT,” different amounts of microcurrents are provided to the skin of the human body to help with the treatment of wounds of different severity.

However, existing technologies, including the above-mentioned Korean registered patent, control the intensity of a microcurrent by identifying the severity of a wound with the naked eye of a practitioner, so there is a lot of room for the subjective judgment by the practitioner, and it is also difficult for a layperson to judge the severity of a wound.

### SUMMARY OF THE INVENTION

Accordingly, the present disclosure has been made keeping in mind the above problems occurring in the related art, and the present disclosure is intended to propose a microcurrent application apparatus for wound healing that can objectively determine the severity or current condition of a wound and apply a suitable microcurrent accordingly.

In order to achieve the objectives of the present disclosure, there is provided a microcurrent application apparatus for wound healing, the apparatus including: a healing pad part attached to a wound site; a pH sensor attached to the healing pad part and configured to measure a pH value of the wound site; at least one electrode attached to the healing pad part and configured to apply a microcurrent to the wound site; and a current controller configured to control at least one of polarity and intensity of the microcurrent applied through the electrode on the basis of the pH value measured by the pH sensor.

Here, the healing pad part may include: an absorbent pad attached to the wound site to absorb exudate generated from the wound site, and an adhesive nonwoven fabric attached to the skin while covering the absorbent pad so that the absorbent pad is fixed to the wound site, wherein the pH sensor and the electrode may be attached to the absorbent pad between the absorbent pad and the adhesive nonwoven fabric.

In addition, the pH sensor may measure a pH value of the exudate generated from the wound site and absorbed by the absorbent pad.

In addition, the current controller may apply a negative polarity through the electrode when the wound site is recognized as an acidic environment on the basis of the pH value measured by the pH sensor, and apply a positive polarity through the electrode when the wound site is recognized as an alkaline environment on the basis of the pH value measured by the pH sensor.

In addition, the electrode may include: a first electrode positioned above the wound site, and a second electrode positioned around the wound site, wherein the current controller may control a polarity of the electrode applied through the first electrode and the second electrode so that a negative polarity or a positive polarity is applied through the first electrode.

In addition, the absorbent pad may include a main absorbent pad, to which the first electrode and the pH sensor are attached and which is applied to the wound site, and an auxiliary absorbent pad, which is electrically separated from the main absorbent pad and is attached to a periphery of the wound site.

With the above configuration, the present disclosure provides a microcurrent application apparatus for wound healing, which can objectively determine the severity or current condition of a wound by detecting a pH value and apply a suitable microcurrent accordingly, thereby maximizing the therapeutic effect.

In addition, the present disclosure provides the microcurrent application apparatus for wound healing, which can switch the polarity of a microcurrent according to whether a pH value is alkaline or acidic, thereby maximizing the therapeutic effect.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a view illustrating the configuration of a microcurrent application apparatus for wound healing according to an embodiment of the present disclosure, and

FIG. 2 is a view illustrating an example in which the microcurrent application apparatus for wound healing according to the embodiment of the present disclosure is attached to a human arm.

### DETAILED DESCRIPTION OF THE INVENTION

The present disclosure may be subject to various modifications and may have multiple embodiments, and thus specific embodiments are illustrated in the drawings and described in detail.

However, this is not intended to limit the present disclosure to a specific embodiment, but should be understood to include all modifications, equivalents, and substitutes included in the spirit and technical scope of the present disclosure.

The terminology used in this application is only used to describe specific embodiments and is not intended to limit the present disclosure. The singular expression includes the plural expression unless the context clearly indicates otherwise. In this application, it should be understood that terms such as “include” or “have” are intended to specify the presence of a feature, number, step, operation, component, part or combination thereof described in the specification, but do not exclude in advance the possibility of the presence or addition of one or more other features, numbers, steps, operations, components, parts or combinations thereof.

Unless otherwise defined, all terms used herein, including technical or scientific terms, have the same meaning as commonly understood by those skilled in the art to which the present disclosure belongs. Terms defined in commonly used dictionaries should be interpreted as having a meaning consistent with their meaning in the context of the relevant art, and will not be interpreted in an idealized or overly formal sense, unless expressly defined in this application.

Hereinafter, embodiments according to the present disclosure will be described in detail with reference to the accompanying drawings.

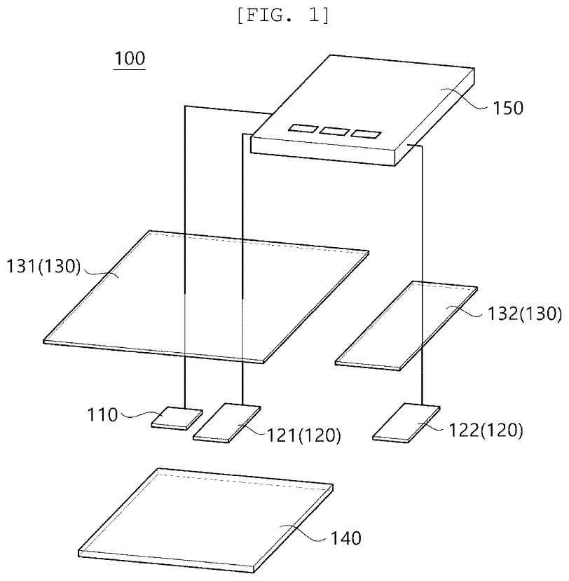

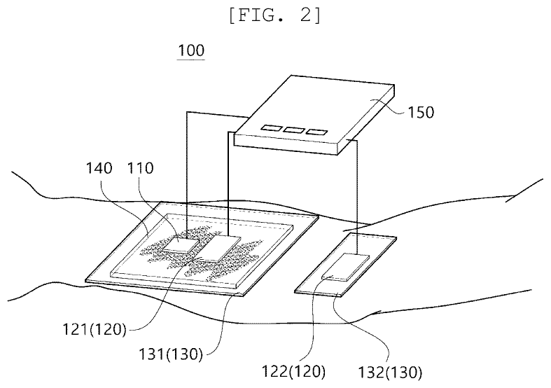

FIG. 1 is a view illustrating the configuration of a microcurrent application apparatus for wound healing according to an embodiment of the present disclosure, and FIG. 2 is a view illustrating an example in which the microcurrent application apparatus for wound healing according to the embodiment of the present disclosure is attached to a human arm.

Referring to FIGS. 1 and 2, the microcurrent application apparatus 100 for wound healing according to the embodiment of the present disclosure may include a healing pad part 130, 140, a pH sensor 110, at least one electrode 120, and a current controller 150.

The healing pad part 130, 140 according to the embodiment of the present disclosure may be attached to a wound site on human skin.

In an embodiment, the healing pad part 130, 140 may include an absorbent pad 140 and an adhesive nonwoven fabric 130.

The absorbent pad 140 according to the embodiment of the present disclosure may be attached to a wound site and absorb exudate generated from the wound site. In addition, the adhesive nonwoven fabric 130 may be attached to the skin in such a manner that the adhesive nonwoven fabric 130 covers the absorbent pad 140 so that the absorbent pad 140 is fixed to the wound site.

The pH sensor 110 according to the embodiment of the present disclosure may be attached to the healing pad part 130, 140. In an embodiment, the pH sensor 110 may be attached to the absorbent pad 140 between the absorbent pad 140 and the adhesive nonwoven fabric 130.

Here, the pH sensor 110 may measure the pH value of a wound site while attached to the absorbent pad 140. In an embodiment, the pH sensor 110 may measure the pH value of exudate generated from the wound site and absorbed by the absorbent pad 140.

The electrode 120 according to the embodiment of the present disclosure may be attached to the healing pad part. In an embodiment, the electrode 120 may be attached to the absorbent pad 140 between the absorbent pad 140 and the adhesive nonwoven fabric 130. In another embodiment, the electrode 120 may be attached to the absorbent pad 140 such that the electrode 120 is positioned between the absorbent pad 140 and the skin so as to be in direct contact with the skin.

Here, the electrode 120 applies a microcurrent to a wound site while being attached to the healing pad part. In an embodiment of the present disclosure, the electrode 120 includes a pair of electrodes and applies a microcurrent from the current controller 150.

The current controller 150 according to the embodiment of the present disclosure may control at least one of the polarity and intensity of the microcurrent applied through the electrode 120 on the basis of the pH value measured by the pH sensor 110.

In an embodiment, the current controller 150 may apply a negative polarity through the electrode 120 when the wound site is recognized as an acidic environment on the basis of the pH value measured by the pH sensor 110. In addition, the current controller 150 may apply a positive polarity through the electrode 120 when the wound site is recognized as an alkaline environment on the basis of the pH value measured by the pH sensor 110.

To this end, the electrode 120 may include a first electrode 121 positioned above the wound site and a second electrode 122 positioned around the wound site. In addition, the current controller 150 may control the polarity of the electrode 120 applied through the first electrode 121 and the second electrode 122 so that a negative polarity or a positive polarity is applied through the first electrode 121.

Here, the first electrode 121 is attached to the absorbent pad 140, and the second electrode 122 is attached to the adhesive nonwoven fabric 130, so that the first electrode 121, the skin, and the second electrode 122 have a structure in which they are electrically connected to each other.

In an embodiment, the adhesive nonwoven fabric 130 may include a first nonwoven fabric 131 for fixing the absorbent pad 140 to the skin and a second nonwoven fabric 132 attached to the skin, with the second electrode 122 attached to the second nonwoven fabric 132.

This allows the current controller to control the polarity applied to the first electrode 121 and the second electrode 122.

Hereinafter, the principle and effect of a process by which the microcurrent application apparatus 100 for wound healing according to the embodiment of the present disclosure controls polarity on the basis of the pH value will be described.

First, in a skin wound, collagen plays an important role in a wound healing process. Collagen is an important protein that forms the structural foundation of the skin and is essential for forming new tissue at the wound site. When a wound occurs, collagen is formed to form a strong foundation at the wound site and allows cells to settle on the foundation.

In the wound healing process, collagen promotes the growth and migration of new cells. In particular, collagen fibers help cells rapidly fill and regenerate the wound site, playing an important role in the skin regeneration process.

In addition, collagen increases the strength of the wound site. As the wound heals, collagen fibers gradually become stronger, helping the skin return to a state closer to an original condition thereof.

Here, when excessive collagen production occurs during the wound healing process, scar tissue may form. The collagen produced at this time may be structurally different from that of normal skin, which leads to the formation of a scar.

Collagen has the characteristic that the charge state of amino acids changes depending on the surrounding pH environment. In a low pH environment, collagen has a positive charge (+), which is advantageous for activating cations such as Cu2+ and Ca2+. Conversely, in a high pH environment, collagen has a negative charge (−), and the activity of cations decreases.

In the present disclosure, the polarity of a microcurrent is controlled according to the pH state of a wound by utilizing the characteristics of collagen as described above.

Collagen is a triple helix protein primarily composed of glycine, proline, and hydroxyproline.

Acidic amino acids, such as glutamic acid and aspartic acid, carry a negative charge, while basic amino acids, such as lysine and arginine, carry a positive charge.

Collagen may be ionic because the charge state of its amino acids changes depending on the pH of the surrounding environment. That is, it carries a positive charge (+) at low pH and a negative charge (−) at high pH.

When a wound becomes infected, its pH may change to alkaline (7 or higher). In an alkaline environment, wound healing is delayed and bacterial growth is promoted. Therefore, it is important to appropriately control the pH of the wound area to maintain an acidic environment (pH 4-6).

In an infected state, the wound exhibits a high pH, that is, an alkaline environment, and due to the infection, cations such as Ca²⁺, Na⁺, and Mg²⁺, along with alkaline byproducts, may accumulate.

Here, when a positive polarity is applied in the alkaline environment of the wound site, it may repel surrounding cations, such as Ca²⁺, Na⁺, and Mg²⁺, thereby creating an acidic environment that inhibits bacterial growth, and lower the concentration of alkaline substances to induce some acidic substances (H⁺).

When a negative polarity is applied, cations, especially Ca²⁺ and Na⁺, are attracted toward the cathode, increasing ion concentration necessary for the wound healing process, thereby promoting cell regeneration and tissue recovery.

Accordingly, in the present disclosure, the current controller 150 applies a negative polarity when the pH condition of the wound site is a low acidic environment on the basis of a pH value measured by the pH sensor 110, thereby activating cations such as Cu²⁺ and Ca²⁺.

On the other hand, the current controller 150 applies a positive polarity when the wound site has a high pH, that is, an alkaline environment on the basis of the pH value measured by the pH sensor 110, thereby suppressing the activity of cations.

Through this process, the wound healing environment may be optimized.

Although some embodiments of the present disclosure have been illustrated and described, those skilled in the art to which the present disclosure pertains will appreciate that various modifications may be made without departing from the principles or spirit of the present disclosure. The scope of the present disclosure is defined by the appended claims and their equivalents.

What is claimed is:

1. A microcurrent application apparatus for wound healing, the apparatus comprising:
a healing pad part attached to a wound site;
a pH sensor attached to the healing pad part and configured to measure a pH value of the wound site;
at least one electrode attached to the healing pad part and configured to apply a microcurrent to the wound site; and
a current controller configured to control at least one of polarity and intensity of the microcurrent applied through the electrode on the basis of the pH value measured by the pH sensor.

2. The apparatus of claim 1, wherein the healing pad part comprises:
an absorbent pad attached to the wound site to absorb exudate generated from the wound site, and
an adhesive nonwoven fabric attached to the skin while covering the absorbent pad so that the absorbent pad is fixed to the wound site,
wherein the pH sensor and the electrode are attached to the absorbent pad between the absorbent pad and the adhesive nonwoven fabric.

3. The apparatus of claim 2, wherein the pH sensor measures a pH value of the exudate generated from the wound site and absorbed by the absorbent pad.

4. The apparatus of claim 2, wherein the current controller applies a negative polarity through the electrode when the wound site is recognized as an acidic environment on the basis of the pH value measured by the pH sensor, and
applies a positive polarity through the electrode when the wound site is recognized as an alkaline environment on the basis of the pH value measured by the pH sensor.

5. The apparatus of claim 4, wherein the electrode comprises:
a first electrode positioned above the wound site, and
a second electrode positioned around the wound site,
wherein the current controller controls a polarity of the electrode applied through the first electrode and the second electrode so that a negative polarity or a positive polarity is applied through the first electrode.

6. The apparatus of claim 5, wherein the first electrode is attached to the absorbent pad, and the second electrode is attached to the adhesive nonwoven fabric.
